SEQUENCE LISTING (5 sequences)

SEQ ID NO: 1 - protein, 69 aa, Artificial Sequence
  Description of Artificial Sequence Illustrative SGP sequence
AAAAAAWAAAAGPNGLYLKKKLLKKLLKLLGNPGLKLYKKLLKKLLLKLGNPGLLKLYKKLLKKLLKLL

SEQ ID NO: 2 - protein, 69 aa, Artificial Sequence
  Description of Artificial Sequence Illustrative SGP variant
AAAAAAWAAAAGGGGLKLLKKLYKKLLKLLGGGGLKLLKKYLKKLLKLLGGGGLLKLLKKYLKKLLKLL

SEQ ID NO: 3 - protein, 69 aa, Artificial Sequence
  Description of Artificial Sequence Illustrative SGP variant
AAAAAAWAAAAGNPGLELLKKLYKKLLELLGNPGLELLKKYLKKLLELLGNPGLLELLKKYLKKLLELL

SEQ ID NO: 4 - protein, 69 aa, Artificial Sequence
  Description of Artificial Sequence Illustrative SGP variant
LLLLLLWLLLLGPNGLKLYKKLLKKLLKLLGNPGLKLYKKLLKKLLLKLGNPGLLKLYKKLLKKLLKLL

SEQ ID NO: 5 - protein, 14 aa, Artificial Sequence
  Description of Artificial Sequence Illustrative control peptide
DLSLARLATARLAI

(19) United States
(12) Patent Application Publication
(10) Pub. No.: US 2005/0256040 A1
(43) Pub. Date: Nov. 17, 2005

(54) Artificially designed pore-forming proteins with anti-tumor effects

(75) Inventors: Dale E. Bredesen, Rancho Santa Fe, CA (US); H. Michael Ellerby, Novato, CA (US)

Correspondence Address: FOLEY & LARDNER, P.O. BOX 80278, SAN DIEGO, CA, 92138-0278, US

(73) Assignee: Buck Institute

(21) Appl. No.: 10/846,479

(22) Filed: May 14, 2004

(51) Int. Cl. A61K 38/17

(52) U.S. Cl. 514/12

(57) ABSTRACT

Protein engineering is an emerging area that has expanded the understanding in the art of protein folding and laid the groundwork for the creation of unprecedented structures with unique functions. The first native-like pore-forming protein, small globular protein (SGP), has previously been designed. It has now been discovered that this artificially engineered protein, and analogs and homologs thereof, have membrane-disrupting properties and anti-tumor activity in several cancer animal models. A mechanism for the selectivity of SGP toward cell membranes in tumors is proposed and validated herein, thereby confirming the proposed mechanism of action. Thus, SGP is established herein as the prototype for a new class of artificial proteins designed for therapeutic applications.

### FIELD OF THE INVENTION

The present invention relates to methods for disrupting biological membranes, compounds useful therefore, and methods for the use thereof.

### BACKGROUND OF THE INVENTION

The tendency of amphipathic peptides to assemble in aqueous solution and of the β-turn to form a loop has been successfully employed to design coiled-coil proteins (see, for example, DeGrado, et al., (1989) Science 243, 622-628; Betz, et al., (1997) Biochemistry 36, 12450-2458; and Bryson, et al., (1998) Prot. Sci. 7, 1404-1414), various helix bundle proteins (see, for example, Walsh, et al., (1999) Proc. Natl. Acad. Sci. U.S.A. 96, 5486-5491; Hecht, et al., (1990) Science 249, 884-891; Dekker, et al., (1993) Nature 362, 852-855; Zhou, et al., (1992) J. Biol. Chem. 267, 2664-2670; Kamtekar, et al., (1993) Science 262, 1680-1685; and Monera, et al., (1996) J. Biol. Chem. 271, 3995-4001), and β-structural proteins (see, for example, Quinn, et al., (1994) Proc. Natl. Acad. Sci. U.S.A. 91, 8747-8751; and Hecht, M. H. (1994) Proc. Natl. Acad. Sci. U.S.A. 91, 8729-8730). De novo design of proteins with biological function, such as hemebinding, catalysis, or the formation of a membrane pore or channel, is perhaps the most challenging goal of peptide chemistry (see, for example, Tuchscherer, et al., (1998) Biopolymers 47, 63-73; Handel, et al., (1993) Science 261, 879-885; Lazar, et al., (1997) Prot. Sci. 6, 1167-1178; Rojas, et al., (1997) Prot. Sci. 6, 2512-2524; Farinas, E., and Regan, L. (1998) Prot. Sci. 7, 1939-1946; Tommos, et al., (1999) Biochem. 38, 9495-9507; Corey, M. J., and Corey, E. (1996) Proc. Natl. Acad. Sci. U.S.A. 93, 11428-11434; and Bayley, H. (1999) Curr. Opin. Biotechnol. 10, 94-103).

Much has been done recently in terms of designing membrane proteins that are correctly incorporated into membranes. However, relatively few attempts have been made to design proteins capable of disrupting membranes and subsequently causing cell death in vivo (see, for example, Bayley, H. (1999) Curr. Opin. Biotechnol. 10, 94-103; and Mingarro, et al., (1997) Trends Biotechnol. 15, 432-437).

Small globular protein (SGP) is a 69-amino acid, 4-helix bundle protein, composed of 3 amphipathic helices, which consist of Leu and Lys residues and surround a single hydrophobic helix consisting of Ala residues, which create a pocket-like structure (see FIGS. 1A and 1B) (see, for example, Lee, et al., (1997) Biochem. 36, 3782-3791; and Matsumoto, et al., (2001) Biopolymers 56, 96-108). SGP is monomeric in solution and denatures in a highly cooperative manner, characteristic of native globular-like proteins. SGP was conceived and designed based on the structure of the colicin family of bacteriocins (see, for example, Konisky, J. (1982) Ann. Rev. Microbiol. 36, 125-144; van der Goot, et al., (1991) Nature 354, 408-410; Zakharov et al., (1998) Proc. Natl. Acad. Sci., U.S.A. 95, 4282-4287; and Mel, S. F., and Stroud, R. M. (1993) Biochem. 32, 2082-2089). Although most naturally occurring, pore-forming proteins maintain their tertiary structure when disrupting membranes, the colicins undergo a spontaneous transition from a native folded state in solution to an open umbrella-like state in membranes. SGP was designed to mimic this membrane insertion mechanism, which was confirmed in synthetic bilayers, where SGP formed a uniform size pore (14 pS) (see, for example, Lee, et al., (1997) supra). It is still not known whether SGP oligomerizes to form a channel.

Given that SGP forms pores in synthetic membranes, it remains of interest to determine whether SGP could disrupt biological membranes at the cellular level and whether it could be used successfully in vivo as an anti-tumor agent. It is also of interest to determine whether SGP shows any selectivity toward tumor cell lines in vitro and in vivo.

### SUMMARY OF THE INVENTION

Protein engineering is an emerging area that has expanded the understanding in the art of protein folding and laid the groundwork for the creation of unprecedented structures with unique functions. The first native-like pore-forming protein, small globular protein (SGP), has previously been designed. It has now been discovered that this artificially engineered protein, and functional derivatives thereof, have membrane-disrupting properties and anti-tumor activity in several cancer animal models. A mechanism for the selectivity of SGP toward cell membranes in tumors is proposed and validated herein, thereby confirming the proposed mechanism of action. Thus, SGP is established herein as the prototype for a new class of artificial proteins designed for therapeutic applications.

SGP represents a novel class of anti-cancer proteins whose therapeutic effects can be optimized by amino acid substitution and by altering helical domain length and hydrophobicity (see Dathe, et al. (1997) FEBS Lett. 403, 208-212). Although SGP is a nonspecific membrane-disrupting agent, it is selective in the sense that the disruption is limited in vivo. Unlike detergents, which solubilize membranes, SGP physically disrupts membrane architecture, leading to cell lysis. This explains the lack of SGP toxicity when the protein is injected sub-cutaneously or intradermally. Recently published data (see Matsumoto, et al. (2001) Biopolymers 56, 96-108) also suggest that the lipid membrane-disruption properties of SGP are responsible for the anti-tumor activity of the agent.

Accordingly, reported herein is one of the first examples of a pore-forming peptide or protein, natural or synthetic, being applied successfully to treat established human tumor xenografts. It is important to emphasize that SGP is not a bacterial toxin, although such agents (or their natural or recombinant form) have been extensively explored as anti-cancer therapies (see, for example, Pastan, et al. (1997) in Encyclopedia of Cancer (Bertino, J., ed) 2nd Ed., pp. 1303-1313, Academic Press, New York). Several pore-forming peptides and proteins have been shown to have moderate efficacy in killing tumor cells in vitro, yet very limited anti-tumor effects were seen in vivo. The anti-bacterial peptides magainin (and synthetic derivatives) (see, for example, Ohsaki, et al. (1992) Cancer Res. 52, 3534-3538), cecropin (and synthetic derivatives; see, for example, Moore, et al. (1994) Peptide Res. 7, 265-269), granulysin (see Gamen, et al. (1998) J. Immunol. 161, 1758-1764), and NK-lysin (see Andersson, et al. (1995) EMBO J. 14, 1615-1625) are toxic to tumor cells in culture. The pore-forming protein verotoxin 1 (a colicin) has also been shown to have a toxic effect on tumor cells in vitro. Magainin, cecropin, and verotoxin 1 also had limited efficacy in vivo in mice bearing murine tumors (see, for example, Ohsaki, et al. (1992) Cancer Res. 52, 3534-3538; Moore, et al. (1994) Peptide Res. 7, 265-269; and Farkas-Himsley, et al. (1995) Proc. Natl. Acad. Sci. U.S.A. 92, 6996-7000).

Cytotoxic agents developed within the past few decades have been based on naturally existing compounds, synthetic peptides, or protein fragments representing active membrane-disrupting domains. In contrast to such compounds, SGP is a protein that was artificially created to perform a pre-determined biological function. Moreover, therapeutically significant cell membrane disrupting activity was observed in vivo. SGP activity appears to be restricted to the presence of lipid bilayers in vitro, whereas in vivo its activity appears to be limited to tumors in vivo due to the protective effect of extracellular matrix components. In vitro, SGP shows no selectivity toward normal or malignant cells under the experimental conditions tested. In accordance with the present invention, it is shown that SGP is potentially a valid anti-cancer agent; applications include Kaposi's sarcoma, malignant melanoma of the skin, or palliation for unresectable or metastatic tumors in anatomical sites difficult to treat with other modalities. Moreover, SGP variants in which residues critical for helical structure are altered are inactive, suggesting that the structure of the protein is intrinsically linked to its ability to damage cell membranes.

### BRIEF DESCRIPTION OF THE FIGURES

FIG. 1 collectively presents SGP sequence, schematic representations thereof, and proposed mechanisms of action.

FIG. 1A presents the amino acid sequence of SGP. Hydrophobic leucine and alanine residues are shown in red, and positively charged lysine residues are in green. Loop residues (glycine, proline, and asparagine) are shown in blue, and tyrosine and tryptophan residues are in black.

FIG. 1B presents an helical wheel diagram of SGP.

FIG. 1C illustrates the putative mechanism by which SGP interacts with a cell membrane. (Note red and green colors are reversed in B). In the aqueous phase SGP folds into a globular protein (upper), but in lipid membranes it adopts an inverted umbrella-like structure forming a pore (lower).

FIG. 2 collectively illustrates the use of SGP for treatment of cultured tumor cells.

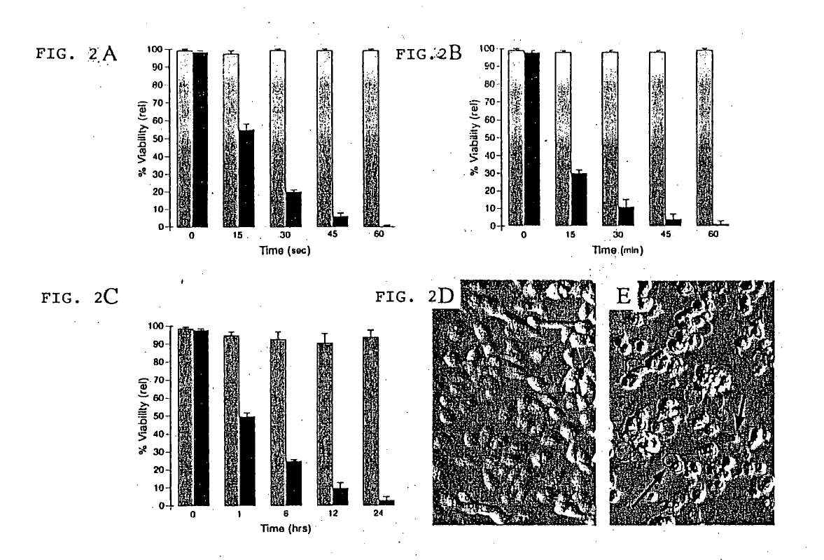

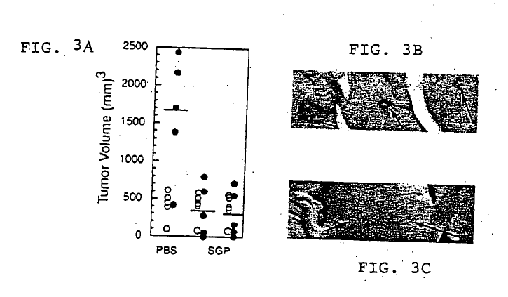

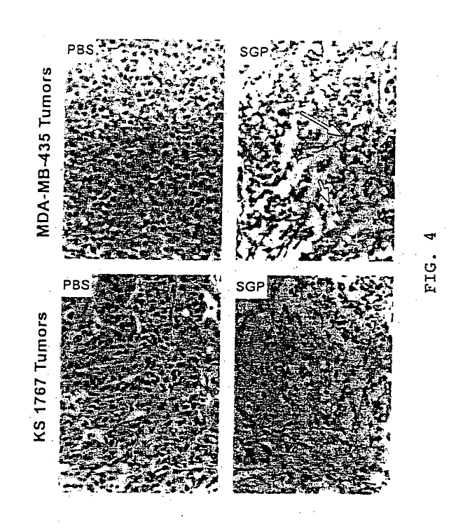

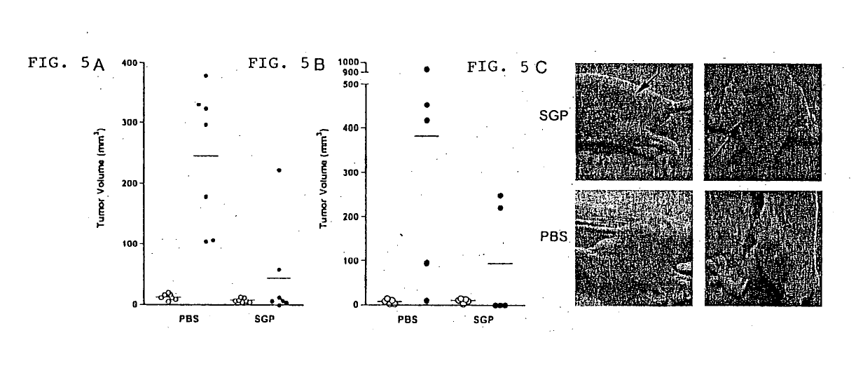

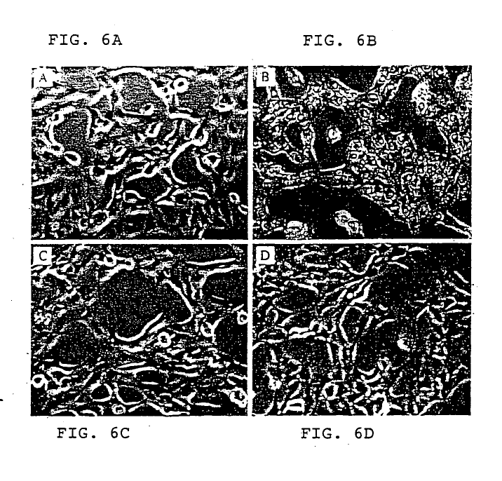

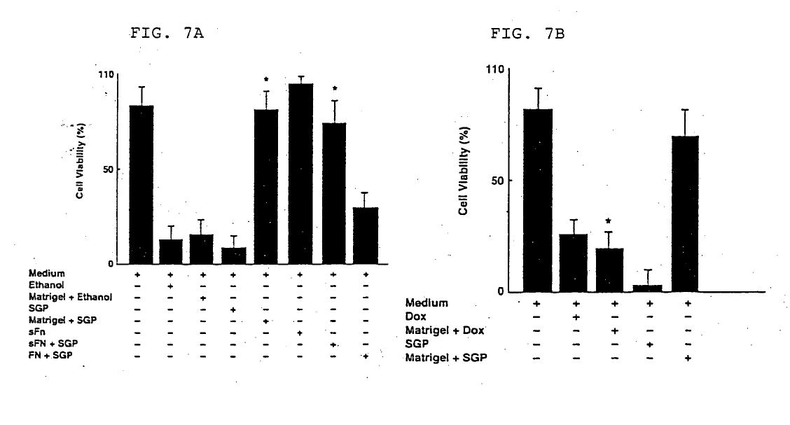

In FIG. 2A, human Kaposi's sarcoma-derived KS1767 cells treated with 10 μM SGP are seen to undergo extremely rapid non-necrotic, non-apoptotic cell death within 60 s (black bars), whereas those treated with 100 μM of negative control peptide DLSLARLATARLAI (SEQ ID NO:xx) are unaffected (scheme bars) (p<0.04).

In FIG. 2B, necrosis is observed in KS1767 cells treated with 10 μM SGP within 60 min (black bars), whereas those treated with 100 μM of negative control peptide are unaffected after 60 min (gray bars) (p<0.03).

In FIG. 2C, apoptosis is observed after treatment with 3 μM SGP over 24 h, whereas cells treated with 100 μM of negative control peptide are unaffected after 24 h (gray bars) (p<0.05).

FIGS. 2D and 2E present Hoffman contrast microscopy of KS1767 cells treated with 100 μM of negative control peptide (FIG. 2D) for 24 h or 3 μM SGP for 24 h (FIG. 2E). Cells with nuclei exhibiting margination and condensation of chromatin and/or nuclear fragmentation (early/mid apoptosis-acridine orange positive) or with compromised plasma membranes (late apoptosis-ethidium bromide positive) were scored as not viable (500 cells per time point were scored in each experiment). Percent viability was calculated relative to untreated cells under all experimental conditions. Classic morphological characteristics of cell death including condensed nuclei (short arrows) and plasma membrane blebbing (long arrows) are evident. Results were reproduced in more than three independent experiments.

FIG. 3 collectively illustrates SGP treatment of nude mice bearing human breast cancer-derived xenografts. Data are shown for human MDA-MB-435-derived breast carcinomas. Mice had tumor volumes ranging from 100 mm³ to 600 mm³ and were divided in similar groups based on matched tumor volumes at the start of the experiment (open circles).

In FIG. 3A, SGP-treated tumors are observed to be smaller than controls (PBS-treated or SGPtreated tumor volumes at the end of the experiment are represented as closed circles). Differences in tumor volumes at 8 weeks are shown (t-test, p<0.05). A total of 10 mice received SGP.

In FIG. 3B, representative pictures are presented for tumors after 4 weekly treatments with SGP at 40 μl/week, n=5 for each experimental group. The volume of the PBS-treated tumor is 400 mm³ (left), whereas 100 μM SGP (middle) and 1 mM SGP (right) treated tumors have flattened and virtually disappeared. These three tumors began at volumes of 100 mm³.

In FIG. 3C, the lack of skin toxicity of SGP is illustrated. Subcutaneous injection (40 μl) of 100 μM SGP (left injection sight, arrow) and of PBS (right injection site, arrow) demonstrates that SGP is relatively non-toxic to normal skin. Results represented presented in FIG. 3C were reproduced in eight independent experiments.

FIG. 4 collectively demonstrates that SGP-treated tumors undergo widespread cell death. Histopathological tissue sections of human tumor xenografts harvested at 8 weeks after treatment initiation are shown. Tissue sections from human MDA-MB-435-derived breast carcinoma xeno-grafts from nude mice treated with PBS-treated tumor tissue but with 100 μM SGP, show extensive apoptosis with many evident condensed nuclei (short arrows) and an intact extra-cellular matrix (long arrows); n=7 for each experimental group. Tissue sections from human KS1767-derived Kaposi's sarcoma xenografts in nude mice had a similar outcome, a representative image of a PBS-treated tumor, and a tumor treated with SGP are shown.

FIG. 5 collectively demonstrates the benefit of SGP treatment of nude mice bearing human prostate and lung cancer xenografts. Data are shown for human PC3-derived prostate carcinoma and H358 lung carcinoma. Tumor cells were implanted on the flank at the start of the experiments. Mice were divided in similar groups based on matched tumor volumes at the start of the experiment (open circles).

In FIG. 5A, SGP-treated PC-3 tumors are observed to be smaller than control PBS-treated tumors. Differences in tumor volumes at 10 weeks are shown (t test, p<0.05).

In FIG. 5B, SGP-treated H358 tumors are observed to be smaller than control PBS-treated tumors. Differences in tumor volumes at 9 weeks are shown (t test, p<0.05).

In FIG. 5C, representative pictures of tumors after 6 weekly treatments at 40 μl/week (see “Experimental Procedures”); n=7 for each experimental group. SGP-treated tumors, as indicated, have disappeared. In FIG. 5A, SGP-treated tumors are observed to be smaller than controls (PBS-treated or SGP-treated tumor volumes at the end of the experiment are represented as closed circles).

FIG. 6 collectively illustrates the effect of SGP treatment of cultured tumor cells in the presence or absence of matrigel or polymeric fibronectin. Treatment of KS1767 cells with 1 mM SGP decreases cell viability and leads to condensed nuclei and plasma cell membrane blebbing (see FIG. 6B), whereas cells treated with 1 mM of SGP in the presence of matrigel remain unaffected after 60 min (see FIG. 6D). KS1767 cells without (see FIG. 6A) or with a layer of matrigel (see FIG. 6C) remained healthy for as long as 48 h. Results were reproduced in four independent experiments.

FIG. 7 collectively presents the results of cytotoxic assays in vitro and the effects of matrigel.

In FIG. 7A, KS1767 cells were exposed to doxorubicin or SGP in the presence or absence of matrigel for 24 h. Cell viability (%) was evaluated at 24 h after no treatment (medium or matrigel alone), or incubation with SGP or doxorubicin (20 μg/well), as indicated. In contrast to SGP, doxorubidin decreased cell viability (*, p<0.01) in the presence of matrigel. Shown are S.E. obtained from triplicate wells. Results were reproduced in four independent experiments.

In FIG. 7B, KS1767 cells were exposed to SGP in the presence or absence of polymeric fibronectin. In contrast to cells exposed to ethanol, cells treated with 1 mM of SGP in the presence of polymeric fibronectin (sFN) remain unaffected (*, p<0.01). Cell viability (%) was evaluated morphologically. Shown are S.E. obtained from triplicate wells. Results were reproduced in three independent experiments.

### DETAILED DESCRIPTION OF THE INVENTION

In accordance with the present invention, there are provided methods for disrupting a biological membrane, said methods comprising contacting said membrane with an effective amount of small globular protein (SGP), or functional derivatives thereof.

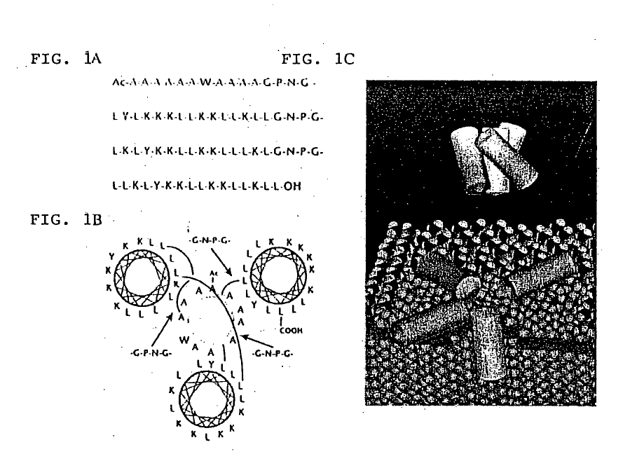

As used herein, “small globular protein” (SGP) refers to an approximately 69-amino acid, 4-helix bundle protein, composed of 3 amphipathic helices, which consist of Leu and Lys residues and surround a single hydrophobic helix consisting of Ala residues, which create a pocket-like structure (see FIGS. 1A and 1B) (see, for example, Lee, et al., (1997) Biochem. 36, 3782-3791; and Matsumoto, et al., (2001) Biopolymers 56, 96-108). SGP is monomeric in solution and denatures in a highly cooperative manner, characteristic of native globular-like proteins. SGP has the following amino acid sequence:

```
(SEQ ID NO:1)
Ac-Ala-Ala-Ala-Ala-Ala-Ala-Trp-Ala-Ala-Ala-Ala-Gly-Pro-Asn-
                                        α-1
Gly-Leu-Tvr-Leu-Lys-Lys-Lys-Leu-Leu-Lys-Lys-Leu-Leu-Lys-
                                                                α-2
Leu-Leu-Gly-Asn-Pro-Gly-Leu-Lys-Leu-Tyr-Lys-Lys-Leu-Leu-
                                                                                          α-3
Lys-Lys-Leu-Leu-Leu-Lys-Leu-Gly-Asn-Pro-Gly-Leu-Leu-Lys-
Leu-Tyr-Lys-Lys-Leu-Leu-Lys-Lys-Leu-Leu-Lys-Leu-Leu-COOH
                                                    α-4
```

As noted above, functional derivatives of SGP are also within the scope of the present invention. The term “functional derivative” indicates a chemically modified version, an analog, or a homolog of a compound that retains a biological function of interest of that compound for any given application. In the case of polypeptides, chemical modification may include, by way of non-limiting example, adding chemical groups to a compound (e.g., glycosylation, phosphorylation, thiolation, pegylation, etc.), eliminating parts of a compound that do not impact the function of interest (preparing a truncated form of a protein that retains an activity of interest, e.g., Klenow fragment), changing sets of one or more amino acids in the polypeptide (preparing muteins); analogs are exemplified by peptidomimetics; and homologs are polypeptides from other species of animals that retain biological activity (e.g., human and porcine insulin, human and salmon calcitonin, etc.) or intraspecies isomers of a polypeptide (protein “families” such as the cytochrome P450 family).

Exemplary variants of SGP contemplated for use herein include

SPG-G:

(SEQ ID NO:2)

Ac-Ala-Ala-Ala-Ala-Ala-Ala-Trp-Ala-Ala-Ala-Ala-
Gly-Gly-Gly-Gly-Leu-Lys-Leu-Leu-Lys-Lys-Leu-Tyr-
Lys-Lys-Leu-Leu-Lys-Leu-Leu-Gly-Gly-Gly-Gly-Leu-
Lys-Leu-Leu-Lys-Lys-Tyr-Leu-Lys-Lys-Leu-Leu-Lys-
Leu-Leu-Gly-Gly-Gly-Gly-Leu-Leu-Lys-Leu-Leu-Lys-
Lys-Tyr-Leu-Lys-Lys-Leu-Leu-Lys-Leu-Leu-COOH,
SGP-E:

(SEQ ID NO:3)

Ac-Ala-Ala-Ala-Ala-Ala-Ala-Trp-Ala-Ala-Ala-Ala-
Gly-Asn-Pro-Gly-Leu-Glu-Leu-Leu-Lys-Lys-Leu-Tyr-
Lys-Lys-Leu-Leu-Glu-Leu-Leu-Gly-Asn-Pro-Gly-Leu-
Glu-Leu-Leu-Lys-Lys-Tyr-Leu-Lys-Lys-Leu-Leu-Glu-
Leu-Leu-Gly-Asn-Pro-Gly-Leu-Leu-Glu-Leu-Leu-Lys-
Lys-Tyr-Leu-Lys-Lys-Leu-Leu-Glu-Leu-Leu-COOH,
SGP-L:

(SEQ ID) NO:4)

Ac-Leu-Leu-Leu-Leu-Leu-Leu-Trp-Leu-Leu-Leu-Leu-
Gly-Pro-Asn-Gly-Leu-Lys-Leu-Tyr-Lys-Lys-Leu-Leu-
Lys-Lys-Leu-Leu-Lys-Leu-Leu-Gly-Asn-Pro-Gly-Leu-
Lys-Leu-Tyr-Lys-Lys-Leu-Leu-Lys-Lys-Leu-Leu-Leu-
Lys-Leu-Gly-Asn-Pro-Gly-Leu-Leu-Lys-Leu-Tyr-Lys-
Lys-Leu-Leu-Lys-Lys-Leu-Leu-Lys-Leu-Leu-COOH,

and the like.

A polypeptide may be substantially related but for a conservative variation, such polypeptides being encompassed by the invention. A conservative variation denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like. The term “conservative variation” also includes the use of a substituted amino acid in place of an unsubstituted parent amino acid provided that antibodies raised to the substituted polypeptide also immunoreact with the unsubstituted polypeptide.

Modifications and substitutions are not limited to replacement of amino acids. For a variety of purposes, such as increased stability, solubility, or configuration concerns, one skilled in the art will recognize the need to introduce, (by deletion, replacement, or addition) other modifications. Examples of such other modifications include incorporation of rare amino acids, dextra-amino acids, glycosylation sites, cytosine for specific disulfide bridge formation. The modified peptides can be chemically synthesized, or the isolated gene can be site-directed mutagenized, or a synthetic gene can be synthesized and expressed in bacteria, yeast, baculovirus, tissue culture and so on.

The term “variant” refers to polypeptides modified at one or more amino acid residues yet still retain the biological activity of SGP. Variants can be produced by any number of means known in the art, including, for example, methods such as, for example, error-prone PCR, shuffling, oligonucleotide-directed mutagenesis, assembly PCR, sexual PCR mutagenesis, and the like, as well as any combination thereof.

By “substantially identical” or “highly conserved” is meant a polypeptide or nucleic acid exhibiting at least 50%, preferably 60%, more preferably 70%, more preferably 80%, more preferably 85%, more preferably 90%, and most preferably 95% homology to a reference amino acid or nucleic acid sequence.

Sequence homology and identity are often measured using sequence analysis software (e.g., Sequence Analysis Software Package of the Genetics Computer Group, University of Wisconsin Biotechnology Center, 1710 University Avenue, Madison, Wis. 53705). The term “identity” in the context of two or more nucleic acids or polypeptide sequences, refers to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. The term “homology” in the context of two or more nucleic acids or polypeptide sequences, refers to two or more sequences or subsequences that are homologous or have a specified percentage of amino acid residues or nucleotides that are homologous when compared and aligned for maximum correspondence over a comparison window or designated region as measured using any number of sequence comparison algorithms or by manual alignment and visual inspection. Programs as mentioned above allow for amino acid substitutions with similar amino acids matches by assigning degrees of homology to determine a degree of homology between the sequences being compared.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequence for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Person & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (J. Roach, http://weber.u.Washington.edu/˜roach/human_genome_progress 2.html) (Gibbs, 1995). Several databases containing genomic information annotated with some functional information are maintained by different organization, and are accessible via the internet, for example, http://wwwtigr.org/tdb; http://www.genetics.wisc.edu; http://genome-www.stanford.edu/˜ball; http://hiv-web.lanl.gov; http://www.ncbi.nlm.nih.gov; http://www.ebi.ac.uk; http://Pasteur.fr/other/biology; and http://www.genome.wi.mit.edu.

One example of a useful algorithm is BLAST and BLAST 2.0 algorithms, which are described in Altschul et al., Nucl. Acids Res. 25:3389-3402 (1977), and Altschul et al., J. Mol. Biol. 215:403-410 (1990), respectively. Software for performing BLAST analyses is publicly available through the National Center for Biotechnology Information (http://www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. USA 90:5873 (1993)). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001.

In one embodiment, protein and nucleic acid sequence homologies are evaluated using the Basic Local Alignment Search Tool (“BLAST”) In particular, five specific BLAST programs are used to perform the following task:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) TBLASTN compares a query protein sequence against a nucleotide sequence database translated in all six reading frames (both strands); and
(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445 (1992); Henikoff and Henikoff, Proteins 17:49-61 (1993)). Less preferably, the PAM or PAM250 matrices may also be used (see, e.g., Schwartz and Dayhoff, eds., Matrices for Detecting Distance Relationships: Atlas of Protein Sequence and Structure, Washington: National Biomedical Research Foundation (1978)). BLAST programs are accessible through the U.S. National Library of Medicine, e.g., at www.ncbi.nlm.nih.gov.

The parameters used with the above algorithms may be adapted depending on the sequence length and degree of homology studied. In some embodiments, the parameters may be the default parameters used by the algorithms in the absence of instructions from the user.

As used herein, “effective amount” refers to levels of compound sufficient to disrupt the normal structure of a biological membrane. Such a concentration typically falls in the range of about 10 nM up to 2 μM; with concentrations in the range of about 100 nM up to 500 nM being preferred. Since the activity of different compounds described herein may vary considerably, and since individual subjects may present a wide variation in severity of symptoms, it is up to the practitioner to determine a subject's response to treatment and vary the dosages accordingly.

As used herein, “biological membrane” refers to the organized assemblies that surround cells. Biological membranes typically comprise proteins and lipids, especially phospholipids.

In accordance with another embodiment of the present invention, there are provided methods for disrupting the membrane architecture of a cell, said methods comprising contacting said cell with an amount of small globular protein (SGP), or functional derivatives thereof, effective to disrupt the membrane architecture thereof.

As used herein, “the membrane architecture of a cell” refers to the three-dimensional relationship of the various components of a cell membrane.

In accordance with yet another embodiment of the present invention, there are provided methods for inducing cell lysis, said method comprising contacting said cell with an amount of small globular protein (SGP), or functional derivatives thereof, effective to induce lysis thereof.

As used herein, “cell lysis” refers to the process of disrupting the cell wall and ultimate destruction of the cell.

In accordance with still another embodiment of the present invention, there are provided methods for selectively disrupting a cell membrane, said method comprising contacting said membrane in the absence of extracellular matrix with an amount of small globular protein (SGP), or functional derivatives thereof, effective to disrupt said membrane.

As used herein, “selectively” disrupting a cell membrane refers to the ability to disrupt only cell membranes which present in a defined environment, such as the absence of extracellular matrix.

In accordance with a further embodiment of the present invention, there are provided methods for treating a tumor in a subject in need thereof, said method comprising administering to said subject an amount of small globular protein (SGP), or functional derivatives thereof, effective to disrupt growth of said tumor.

As used herein, “treating” refers to inhibiting or arresting the development of a disease, disorder or condition and/or causing the reduction, remission, or regression of a disease, disorder or condition. Those of skill in the art will understand that various methodologies and assays may be used to assess the development of a disease, disorder or condition, and similarly, various methodologies and assays may be used to assess the reduction, remission or regression of a disease, disorder or condition.

A wide variety of tumors are contemplated for treatment in accordance with the present invention, e.g., tumors associated with Kaposi's sarcoma, tumors associated with breast carcinoma, tumors associated with malignant melanoma of the skin, tumors associated with prostate cancer, tumors associated with lung cancer, tumors associated with unresectable or metastatic tumors in anatomical sites difficult to treat with other modalities, and the like.

As used herein, “administering” refers to providing a therapeutically effective amount of a compound to a subject, using oral, sublingual, intravenous, subcutaneous, transcutaneous, intramuscular, intracutaneous, intrathecal, epidural, intraoccular, intracranial, inhalation, rectal, vaginal, and the like administration. Administration in the form of creams, lotions, tablets, capsules, pellets, dispersible powders, granules, suppositories, syrups, elixirs, lozenges, injectable solutions, sterile aqueous or non-aqueous solutions, suspensions or emulsions, patches, and the like, is also contemplated. The active ingredients may be compounded with non-toxic, pharmaceutically acceptable carriers including, glucose, lactose, gum acacia, gelatin, mannitol, starch paste, magnesium trisilicate, talc, corn starch, keratin, colloidal silica, potato starch, urea, dextrans, and the like.

The preferred route of administration will vary with the clinical indication. Some variation in dosage will necessarily occur depending upon the condition of the patient being treated, and the physician will, in any event, determine the appropriate dose for the individual patient. The effective amount of compound per unit dose depends, among other things, on the body weight, physiology, and chosen inoculation regimen. A unit dose of compound refers to the weight of compound employed per administration event without the weight of carrier (when carrier is used).

Targeted-delivery systems, such as polymer matrices, liposomes, and microspheres can increase the effective concentration of a therapeutic agent at the site where the therapeutic agent is needed and decrease undesired effects of the therapeutic agent. With more efficient delivery of a therapeutic agent, systemic concentrations of the agent are reduced because lesser amounts of the therapeutic agent can be administered while accruing the same or better therapeutic results. Methodologies applicable to increased delivery efficiency of therapeutic agents typically focus on attaching a targeting moiety to the therapeutic agent or to a carrier which is subsequently loaded with a therapeutic agent.

Various drug delivery systems have been designed by using carriers such as proteins, peptides, polysaccharides, synthetic polymers, colloidal particles (i.e., liposomes, vesicles or micelles), microemulsions, microspheres and nanoparticles. These carriers, which contain entrapped pharmaceutically useful agents, are intended to achieve controlled cell-specific or tissue-specific drug release.

The compounds contemplated for use herein can be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The compounds described herein, when in liposome form can contain, in addition to the compounds described herein, stabilizers, preservatives, excipients, and the like. The preferred lipids are the phospholipids and the phosphatidyl cholines (lecithins), both natural and synthetic. Methods to form liposomes are known in the art. (See, e.g., Prescott, Ed., Methods in Cell Biology, Volume XIV, Academic Press, New York, N.Y., (1976), p 33 et seq.)

Several delivery approaches can be used to deliver therapeutic agents to the brain by circumventing the blood-brain barrier. Such approaches utilize intrathecal injections, surgical implants (Ommaya, Cancer Drug Delivery, 1: 169-178 (1984) and U.S. Pat. No. 5,222,982), interstitial infusion (Bobo et al., Proc. Natl. Acad. Sci. U.S.A., 91: 2076-2080 (1994)), and the like. These strategies deliver an agent to the CNS by direct administration into the cerebrospinal fluid (CSF) or into the brain parenchyma (ECF).

Drug delivery to the central nervous system through the cerebrospinal fluid is achieved, for example, by means of a subdurally implantable device named after its inventor the “Ommaya reservoir”. The drug is injected into the device and subsequently released into the cerebrospinal fluid surrounding the brain. It can be directed toward specific areas of exposed brain tissue which then adsorb the drug. This adsorption is limited since the drug does not travel freely. A modified device, whereby the reservoir is implanted in the abdominal cavity and the injected drug is transported by cerebrospinal fluid (taken from and returned to the spine) to the ventricular space of the brain, is used for agent administration. Through omega-3 derivatization, site-specific biomolecular complexes can overcome the limited adsorption and movement of therapeutic agents through brain tissue.

Another strategy to improve agent delivery to the CNS is by increasing the agent absorption (adsorption and transport) through the blood-brain barrier and the uptake of therapeutic agent by the cells (Broadwell, Acta Neuropathol., 79: 117-128 (1989); Pardridge et al., J. Pharmacol. Experim. Therapeutics, 255: 893-899 (1990); Banks et al., Progress in Brain Research, 91:139-148 (1992); Pardridge, Fuel Homeostasis and the Nervous System, ed.: Vranic et al., Plenum Press, New York, 43-53 (1991)). The passage of agents through the blood-brain barrier to the brain can be enhanced by improving either the permeability of the agent itself or by altering the characteristics of the blood-brain barrier. Thus, the passage of the agent can be facilitated by increasing its lipid solubility through chemical modification, and/or by its coupling to a cationic carrier, or by its covalent coupling to a peptide vector capable of transporting the agent through the blood-brain barrier. Peptide transport vectors are also known as blood-brain barrier permeabilizer compounds (U.S. Pat. No. 5,268,164). Site specific macromolecules with lipophilic characteristics useful for delivery to the brain are described in U.S. Pat. No. 6,005,004.

Other examples (U.S. Pat. No. 4,701,521, and U.S. Pat. No. 4,847,240) describe a method of covalently bonding an agent to a cationic macromolecular carrier which enters into the cells at relatively higher rates. These patents teach enhancement in cellular uptake of bio-molecules into the cells when covalently bonded to cationic resins.

U.S. Pat. No. 4,046,722 discloses anti-cancer drugs covalently bonded to cationic polymers for the purpose of directing them to cells bearing specific antigens. The polymeric carriers have molecular weights of about 5,000 to 500,000. Such polymeric carriers can be employed to deliver compounds described herein in a targeted manner.

Further work involving covalent bonding of an agent to a cationic polymer through an acid-sensitive intermediate (also known as a spacer) molecule, is described in U.S. Pat. No. 4,631,190 and U.S. Pat. No. 5,144,011. Various spacer molecules, such as cis-aconitic acid, are covalently linked to the agent and to the polymeric carrier. They control the release of the agent from the macromolecular carrier when subjected to a mild increase in acidity, such as probably occurs within a lysosome of the cell. The drug can be selectively hydrolyzed from the molecular conjugate and released in the cell in its unmodified and active form. Molecular conjugates are transported to lysosomes, where they are metabolized under the action of lysosomal enzymes at a substantially more acidic pH than other compartments or fluids within a cell or body. The pH of a lysosome is shown to be about 4.8, while during the initial stage of the conjugate digestion, the pH is possibly as low as 3.8.

As employed herein, the phrase “therapeutically effective amount”, when used in reference to compounds contemplated for use in the practice of the present invention, refers to a dose of compound sufficient to provide circulating concentrations high enough to impart a beneficial effect on the recipient thereof. The specific therapeutically effective dose level for any particular patient will depend upon a variety of factors including the disorder being treated, the severity of the disorder, the activity of the specific compound used, the route of administration, the rate of clearance of the specific compound, the duration of treatment, the drugs used in combination or coincident with the specific compound, the age, body weight, sex, diet and general health of the patient, and like factors well known in the medical arts and sciences. Dosage levels typically fall in the range of about 0.001 up to 100 mg/kg/day; with levels in the range of about 0.05 up to 10 mg/kg/day being preferred.

In accordance with a still further embodiment of the present invention, there are provided methods for inducing non-necrotic, non-apoptotic cell death of a cell population, said method comprising contacting said cell population with an amount of small globular protein (SGP), or functional derivatives thereof, effective to induce non-necrotic, non-apoptotic cell death of said cell population.

As used herein, “non-necrotic, non-apoptotic cell death” refers to death of a cell as a result of cause(s) other than injury or programmed cell death.

As used herein, a “cell population” refers to plurality of cells, either of homogeneous genotype and/or phenotype, or heterogeneous genotype and/or phenotype.

In accordance with another embodiment of the present invention, there are provided methods for inducing apoptosis of a cell population, said method comprising contacting said cell population with an amount of small globular protein (SGP), or functional derivatives thereof, effective to induce apoptosis of said cell population.

As used herein, “apoptosis” refers to the biologically programmed death of cells. Apoptosis in mammals and other eukaryotic organisms is a characteristic process of cell death, which can, among its other effects, limit the spread of viruses and other intracellular organisms (see, for example, Hershberger, et al., J Virol 66:5525-33, (1992)). For example, the difference in viral titer during Baculoviral infection with and without apoptosis inhibition is 200-15,000-fold. Thus apoptosis is a mechanism of defense against pathogenic infections.

Apoptosis proceeds by the activation of a group of cysteine proteases called caspases (see, for example, Salvesen and Dixit, Cell 91:443-6, (1997)). One of these, caspase-9, is activated when cytochrome c is released from mitochondria, which may occur with the disruption of the mitochondrial outer membrane (see Zou, et al., J Biol Chem 274:11549-56, (1999)). This cytochrome c release in apoptotic cells may be induced by pro-apoptotic members of the Bcl-2 family, such as Bax and Bid, although the mechanism by which this is achieved is incompletely understood (see Jurgensmeier, et al., Proc Natl Acad Sci USA 95:4997-5002, (1998)).

In accordance with still another embodiment of the present invention, there are provided methods for inhibiting primary tumor growth in a subject in need thereof, said method comprising administering to said subject an amount of small globular protein (SGP), or functional derivatives thereof, effective to inhibit primary tumor growth.

As used herein, “primary tumor growth” refers to the initial site of malignant cell growth, prior to any metastatic spread thereof.

As used herein, a “subject in need thereof” refers to a subject suffering from a condition which can be treated by the above-described methods, e.g., a subject having tumors associated with Kaposi's sarcoma, tumors associated with breast carcinoma, tumors associated with malignant melanoma of the skin, tumors associated with prostate cancer, tumors associated with lung cancer, unresectable tumors in anatomical sites difficult to treat with other modalities, and the like.

In accordance with yet another embodiment of the present invention, there are provided methods for inhibiting metastatic tumor growth in a subject in need thereof, said method comprising administering to said subject an amount of small globular protein (SGP), or functional derivatives thereof, effective to inhibit metastatic tumor growth.

As used herein, “metastatic tumor growth” refers to secondary malignant cell growth, as transferred from a primary malignant site, associated, for example, with Kaposi's sarcoma, breast carcinoma, malignant melanoma of the skin, prostate cancer, lung cancer, metastatic tumors in anatomical sites difficult to treat with other modalities, and the like.

In accordance with a still further embodiment of the present invention, there are provided formulations comprising small globular protein (SGP), or functional derivatives thereof, and a pharmaceutically acceptable carrier therefore.

In accordance with yet another embodiment of the present invention, there are provided non-naturally occurring, pore-forming, anti-neoplastic, 4-helix bundle proteins comprising in the range of about 69-amino acids, wherein said protein forms a pocket-like structure composed of 3 amphipathic helices surrounding a single hydrophobic helix, provided, however, that said anti-neoplastic protein does not have the amino acid sequence set forth in SEQ ID NO:1.

Amphipathic helices contemplated for use in the practice of the present invention are composed largely of Leu and Lys residues. See, for example, SEQ ID NOs:1-4.

Hydrophobic helices contemplated for use in the practice of the present invention are compose largely of Ala residues. See, for example, SEQ ID NOs:1-4.

In accordance with yet another embodiment of the present invention, there are provided formulations comprising an anti-neoplastic protein as described hereinabove and a pharmaceutically acceptable carrier therefore.

The invention will now be described in greater detail by reference to the following non-limiting examples.

### EXAMPLE 1

### Reagents

SGP, SGP-L, and SGP-E were synthesized according to the Fmoc procedure starting from Fmoc-Leu-PEG (polyethylene glycol) resin using a Miligen automatic peptide synthesizer (Model 9050) to monitor the de-protection of the Fmoc group by UV absorbance (see Lee, et al. (1997) Biochem. 36, 3782-3791). After cleavage from the resin by trifluoroacetic acid, the crude peptide obtained was purified by HPLC chromatography with an ODS column, 20×250 mm, with a gradient system of water/acetonitrile containing 0.1% trifluoroacetic acid. Amino acid analysis was performed after hydrolysis in 5.7 M HCl in a sealed tube at 110° C. for 24 h. Analytical data obtained were as follows: Gly, 6.2 (6); Ala, 9.5 (10); Leu, 26.5 (25); Asp, 3.0 (3); Pro, 2.9 (3); Tyr, 3.1 (3); Lys, 18.9 (18). Molecular weight was determined by fast atom bombardment mass spectroscopy using a JEOL JMX-HX100: base peak, 7555.1; calculated for C, 367; H, 639; O, 77; N, 91.H+, 7554.8. Peptide concentrations were determined from the UV absorbance of Trp and three Tyr residues at 280 nm in buffer (e=8000). Gel filtration HPLC chromatography was performed using Tris buffer (10 mM Tris, 150 mM NaCl, pH 5.0 or pH 7.4) on COSMOSIL 5DIOL-300 (Nakalai Tesk, Kyoto, Japan).

### EXAMPLE 2

### Computer Model

The computer-generated model of SGP was made with the program Insight II (Molecular Simulations Inc., San Diego, Calif.) running on an Octane SSE work station (Silicon Graphics, Cupertino, Calif.).

### EXAMPLE 3

### Cell Culture

All cell lines were obtained commercially. The Kaposi's sarcoma-derived cell line KS1767 and the breast carcinoma cell line MDA-MB-435 have been described previously (see, for example, Herndier, et al. (1994) Aids 8, 575-581; Reisbach, et al. (1982) Anticancer Res. 2, 257-260; and Ellerby, et al. (1999) Nat. Med. 5, 1032-1038) and were cultured in 10% fetal bovine serum/Dulbecco's modified Eagle's medium, containing antibiotics.

### EXAMPLE 4

### SGP Effects on Cultured Cells

To evaluate the effects of SGP on cell membranes multiple human cell lines of different origins were treated (see Table I, which presents a comparison of LC50 data for SGP, SGP-L, and SGP-E on a variety of cultured human cell types).

Cell viability was determined by morphology (see Ellerby, et al. (1999) Nat. Med. 5, 1032-1038; and Ellerby, et al. (1997) J. Neurosci. 17, 6165-6178). For viability assays, KS1767 cells were incubated with the concentrations of SGP, SGP-L, SGP-E, or control peptides indicated in the figures and in Table I. Briefly, at the given time points, cell culture medium was aspirated from adherent cells. Cells were then gently washed once with PBS at 37° C. A 20-fold dilution of the dye mixture (100 μg/ml acridine orange and 100 μg/ml ethidium bromide) in PBS was then gently pipetted on the cells and viewed on an inverted microscope (Nikon TE 300). Cells with nuclei exhibiting margination and condensation of chromatin and/or nuclear fragmentation (early/mid apoptosis-acridine orange positive) or with compromised plasma membranes (late apoptosis-ethidium bromide positive) were scored as not viable; 500 cells per time point were scored in each experiment. Percent viability was calculated relative to untreated cells.

TABLE I

| | LC₅₀ at 30 min, μM | | |
|---|---|---|---|
| Cell Line* | SGP | SPG-L | SPG-E |
| KS 1767 | 5 | 60 | 30 |
| | 4 | 60 | 30 |
| PC3 | 2.5 | — | — |
| H358 | 6 | — | — |
| CADMEC | 5 | 60 | 30 |
| HUVEC | 7 | — | — |
| HPAEC | 5 | — | — |
| 293 | 5 | — | — |

The dash marks (—) indicate no data obtained
*KS1767 = Kaposi's sarcoma cells;
PC3 = human prostate cancer cells;
H358 = human lung carcinoma cells;
CADMEC = Cell Applications Dermal Microvessel Endothelial Cells;
HUVEC = Human Umbilical Cord Vascular Endothelial Cells;
HPAEC = Human Pulmonary Artery Endothelial Cells; and
293 = human kidney cells.

Treatment of KS1767 cells with >10 μM SGP led to rapid normecrotic, non-apoptotic cell death, characterized by 100% loss of viability within 60 s (FIG. 2A), as determined by Trypan Blue positivity. Such a rapid response suggests that the plasma membrane has been disrupted. Lowering the concentration of SGP to between 5 and 10 μM led to induction of necrosis (scored morphologically), resulting in almost 100% loss of KS1767 cell viability over 60 min (FIG. 2B). SGP levels below 5 μM led to the induction of apoptosis over a 24-hour period (FIG. 2C), which was confirmed by a caspase-3 activation assay. KS1767 cells were unaffected by a 24 h incubation in 100 μM of a control peptide (FIG. 2D). However, the classic morphological signs of apoptosis, such as nuclear condensation (FIG. 2E, short arrow) and plasma membrane blebbing (FIG. 2E, long arrow), were apparent in KS1767 cells after a 24-hour treatment with 3 μM SGP. Similar results were obtained using different cell lines, including several types of malignant cells (solid tumors and leukemic cell lines) and non-neoplastic cells (including endothelial cells and fibroblasts isolated from multiple organs and cells of glial origin, Table I).

As negative controls, altered forms of SGP (SGP-L and SGP-E) were used. In SGP-L, the central all alanine helix was replaced by an all leucine helix. In SGP-E, lysines have been replaced by glutamic acids, and it had previously been determined that the ability of such analogs to disrupt synthetic membranes is diminished (see Matsumoto, et al., (2001) Biopolymers 56, 96-108). SGP-L and SGP-E were substantially less toxic to mammalian cultured cells (Table I). The LC50 was increased by at least 10-fold in all cell types tested with SGP-L and SGP-E when these inactive versions of the protein were tested. These observations clearly show that the integrity of the SGP helices is required for SGP membrane disrupting activity. Taken together, these data demonstrate that SGP is a potent membrane-disrupting agent, but also that it is not cell-selective and it will affect tumor derived cells as well as normal cells at similar concentrations (˜3 μM).

### EXAMPLE 5

### SGP has Anti-Tumor Activity In Vivo

Given the potent membrane-disrupting activity of SGP, SGP anti-tumor activity was evaluated in nude mice bearing human tumor xenografts. It was thought that direct administration of SGP might reduce tumor volume and retard metastasis.

MDA-MB-435-, KS1767-, PC-3-, and H358-derived human tumor xenografts were established in 2-month-old female or male (according to the tumor type), nude/nude Balb/c mice (Jackson Labs, Bar Harbor, Me.) by administering 106 tumor cells per mouse in a 200 μl volume of serum-free Dulbecco's modified Eagle's medium into the mammary fat pad or on the flank (see Ellerby, et al. (1999) Nat. Med. 5, 1032-1038). The mice were anesthetized with Avertin as described (see Ellerby, et al. (1999) supra.). SGP was administered directly into the center of the tumor mass at a concentration of 100 μM or 1 mM given slowly in 5 μl increments, for a total volume of 40 μl. Measurements of tumors were taken by caliper under anesthesia and used to calculate tumor volume (see Ellerby, et al. (1999) supra.). Animal experimentation was reviewed and approved by the Institutional Animal Care and Use Committee.

In the first set of experiments, tumors were allowed to form after injection of a breast carcinoma cell line (MDA-MD-435) and then treated with local injections of SGP. It was observed that tumor volume was significantly smaller in SGP-treated mice than in the PBS-treated control mice (FIG. 3A). Starting tumor volumes ranged from about 100 mm³ to large sizes of about 600 mm³. Tumor-bearing mice were given four weekly treatments of PBS, or 100 μM or 1 mM SGP (40 μl/treatment given in 5 μl increments). After a 4-week period without treatment, the tumor volumes were measured at 8 weeks. The average tumor volume at the end of the experiment in the SGP-treated groups was 5μ less than the average volume seen in the PBS-treated group (FIG. 3A). There was no difference between the average tumor volumes of the 2 SGP treatment groups. Mice treated with SGP remained tumor-free for up to 4 months after tumor implantation, before being euthanized for histological evaluation. These observations indicate that both primary tumor growth (FIG. 4) and metastases were inhibited. Surgical examination of the tumor sites revealed no sign of tumor cells. Similar results were obtained when xenografts were produced by injection of prostate (FIG. 5A) and lung carcinoma (FIGS. 5, B and C) cell lines. By successfully treating a large number of mice and testing the effects of SGP on several different tumor xenograft models (including carcinomas, sarcomas, and melanomas), the therapeutic properties of SGP were firmly established. The data also show that the anti-tumor effects of SGP are not limited to a specific tumor type. It was also evaluated whether SGP produced adverse side effects such as necrosis when injected under normal skin. Strikingly, in all mice tested, SGP did not produce any surface effect when injected intradermally or sub-cutaneously (FIG. 3C) when compared with mice that did not receive the active form of SGP.

### EXAMPLE 6

### Histology

MDA-MB-435-derived breast carcinoma and KS1767-derived Kaposi sarcoma xenografts and organs were removed, fixed in Bouin solution, embedded in paraffin for preparation of tissue sections, and stained with hematoxylin and eosin (see Ellerby, et al. (1999) supra.).

Histopathological analysis of SGP-treated MDA-MD-435 human breast carcinoma xenografts showed widespread cell death (FIG. 4, upper right panel), as compared with PBS-treated tumors (FIG. 4, upper left panel). Many condensed nuclei were apparent (FIG. 4, upper left panel, short arrows), and there was no effect on the extracellular matrix (FIG. 4B, long arrows). Apoptosis was confirmed by a caspase-3 activation assay (data not shown). It is noteworthy that whereas 100 μM SGP induced almost immediate cell death in vitro that was apparently neither apoptotic nor necrotic, 100 μM SGP induced apoptosis in vivo. Lower concentrations can also be used. SGPtreated human KS1767 Kaposi's sarcoma-derived xenografts showed similar effects (FIG. 4, left and right panels). Histological analysis of the major organs of SGP-treated mice showed no overt pathology, confirming that SGP treatments do not affect sites other than the injected tumor area (data not shown). Thus, SGP has anti-tumor specific effects, without showing any tumor cell-specific effects.

### EXAMPLE 7

### Skin Toxicity

2-month-old female nude mice (Jackson Labs) were anesthetized with Avertin. 10 μl of 100 μM SGP or PBS was injected into the skin. The injected areas were monitored for 2 weeks.

### EXAMPLE 8

### Mechanism of SGP Action and Selectivity Toward Cell Membranes

To determine the mechanisms responsible for selective anti-tumor activity of SGP in vivo, a matrigel assay (to mimic extracellular matrix) was developed.

Cell viability was determined by morphology (see Ellerby, et al. (1999) supra.; and Ellerby, et al. (1997) supra.). KS1767 cells were incubated with SGP at 1 mM in the presence or absence of matrigel or polymeric fibronectin (sFN). The fibronectin polymer was produced as previously described (see Pasqualini, et al. (1996) Nature Med. 2, 1197-1203). Briefly, cell culture medium was aspirated from adherent cells. Cells were then coated with matrigel (gently pipetted on each well to completely coat the entire cell layer), or the fibronectin polymer, and incubated at 37° C. for 10 min. SGP was added and the cells were viewed on an inverted microscope (Nikon TE 300). KS1767 cells were also exposed to doxorubicin (20 μg/well) or SGP in the presence or absense of matrigel for 24 h. Cell viability (%) was evaluated after no treatment (medium or matrigel alone), incubation with SGP or doxorubicin. Cell death was evaluated morphologically (see Ellerby, et al. (1999) supra.; and Ellerby, et al. (1997) supra.), and cell viability was compared relative to untreated controls (no matrigel) or absence of SGP.

In the absence of matrigel, SGP led to severe disruption of cell membranes, resulting in almost 100% loss of viability over 10 min (FIG. 6B). In contrast, in the presence of matrigel, KS1767 cells were unaffected by incubation with 1 mM SGP (FIG. 6D). This loss of membrane disrupting ability in the presence of a thin matrigel layer could account for the lack of SGP toxicity seen in vivo. Ethanol, as shown in FIG. 7A, or cytotoxic drugs such as doxorubicin (FIG. 7B) damaged the cell layer under similar conditions, regardless of the presence of matrigel, which fails to provide protection from the other toxic agents because these other agents more readily diffuse through the matrix. When matrigel was replaced by polymeric fibronectin (sFN) (Pasqualini, R., Bourdoulous, S., Koivunen, E., Woods, V. L., Jr., and Ruoslahti, E. (1996) Nature Med. 2, 1197-1203), another form of matrix, SGP was also ineffective and did not interfere with cell viability (FIG. 7A), whereas ethanol induced massive cell death. Fibronectin alone did not prevent SGP activity and was used as a control.

The observations in this model are consistent with the lack of skin toxicity seen with SGP. It is proposed that the discrepancy between in vitro and in vivo SGP effects (anti-tumor cell activity versus selective anti-tumor activity) results from the potent membrane-disrupting activity of SGP, which is inactivated in the presence of extracellular matrix and connective tissue.

While the invention has been described in detail with reference to certain preferred embodiments thereof, it will be understood that modifications and variations are within the spirit and scope of that which is described and claimed.

1. A method for disrupting a biological membrane, said method comprising contacting said membrane with an effective amount of small globular protein (SGP), or functional derivatives thereof.

2. A method for disrupting the membrane architecture of a cell, said method comprising contacting said cell with an amount of small globular protein (SGP), or functional derivatives thereof, effective to disrupt the membrane architecture thereof.

3. A method for inducing cell lysis, said method comprising contacting said cell with an amount of small globular protein (SGP), or functional derivatives thereof, effective to induce lysis thereof.

4. A method for selectively disrupting a cell membrane, said method comprising contacting said membrane in the absence of extracellular matrix with an amount of small globular protein (SGP), or functional derivatives thereof, effective to disrupt said membrane.

5. A method for treating a tumor in a subject in need thereof, said method comprising administering to said subject an amount of small globular protein (SGP), or functional derivatives thereof, effective to disrupt growth of said tumor.

6. The method of claim 5 wherein said tumor is associated with Kaposi's sarcoma.

7. The method of claim 5 wherein said tumor is associated with breast carcinoma.

8. The method of claim 5 wherein said tumor is associated with malignant melanoma of the skin.

9. The method of claim 5 wherein said tumor is associated with prostate cancer.

10. The method of claim 5 wherein said tumor is associated with lung cancer.

11. The method of claim 5 wherein said tumor is associated with unresectable or metastatic tumors in anatomical sites difficult to treat with other modalities.

12. A method for inducing non-necrotic, non-apoptotic cell death of a cell population, said method comprising contacting said cell population with an amount of small globular protein (SGP), or functional derivatives thereof, effective to induce non-necrotic, non-apoptotic cell death of said cell population.

13. A method for inducing apoptosis of a cell population, said method comprising contacting said cell population with an amount of small globular protein (SGP), or functional derivatives thereof, effective to induce apoptosis of said cell population.

14. A method for inhibiting primary tumor growth in a subject in need thereof, said method comprising administering to said subject an amount of small globular protein (SGP), or functional derivatives thereof, effective to inhibit primary tumor growth.

15. A method for inhibiting metastatic tumor growth in a subject in need thereof, said method comprising administering to said subject an amount of small globular protein (SGP), or functional derivatives thereof, effective to inhibit metastatic tumor growth.

16. A formulation comprising small globular protein (SGP), or functional derivatives thereof, and a pharmaceutically acceptable carrier therefore.

17. A non-naturally occurring, pore-forming, anti-neoplastic, 4-helix bundle protein comprising in the range of about 69-amino acids, wherein said protein forms a pocket-like structure composed of 3 amphipathic helices surrounding a single hydrophobic helix, provided, however, that said anti-neoplastic protein does not have the amino acid sequence set forth in SEQ ID NO:1.

18. An anti-neoplastic protein according to claim 17, wherein said amphipathic helices consist essentially of Leu and Lys residues.

19. An anti-neoplastic protein according to claim 17, wherein said hydrophobic helix consists essentially of Ala residues.

20. A formulation comprising an anti-neoplastic protein according to claim 17 and a pharmaceutically acceptable carrier therefore.
